(19) United States
(12) Patent Application Publication
(10) Pub. No.: US 2006/0024383 A1
(43) Pub. Date: Feb. 2, 2006

(54) Compositions containing policosanol and chromium and/or chromium salts and their pharmaceutical uses

(76) Inventor: Roger Berlin, Mendham, NJ (US)

Correspondence Address: WYETH;PATENT LAW GROUP, 5 GIRALDA FARMS, MADISON, NJ, 07940, US

(21) Appl. No.: 10/900,049

(22) Filed: Jul. 27, 2004

(51) Int. Cl. A61K 33/24; A61K 31/045

(52) U.S. Cl. 424/655; 514/724

(57) ABSTRACT

A composition is provided which contains policosanol and chromium and/or chromium salts and which may be used for treating, preventing and or reducing metabolic syndrome, hypercholesterolemia and hypoglycemia related diseases, total cholesterol, LDL-cholesterol, LDL/HDL ratio, triglycerides, coronary heart disease (heart attacks and strokes), inflammation, deep-vein thrombosis, immunoregulatory diseases, cardiovascular diseases, obesity, insulin resistance, dyslipidemia, raised blood pressure, fatigue, premenstrual syndrome, anxiety, depression and/or neurodegenerative disorders, and/or raising HDL cholesterol and/or lean body mass in humans and animals. The method comprises administering policosanol and chromium and/or chromium salts which together effectively lower blood glucose levels and lower the LDL/HDL cholesterol ratio. Typically, the administered composition includes about 0.1-10:1 parts by weight of policosanol to chromium and/or chromium salts.

### BACKGROUND OF THE INVENTION

1. Field of the Invention

The present invention relates to therapeutic compositions and methods for improving glucose levels, cholesterol levels and lean body mass in humans and animals. More particularly the invention pertains to a therapeutic composition and method for improving fat, protein and carbohydrate metabolism, improving insulin activities, increasing HDL cholesterol levels while reducing triglyceride and serum cholesterol levels, and increasing lean body mass while reducing body fat by administering a biologically active mixture of high purity, high molecular weight straight chain primary aliphatic alcohols (referred to collectively herein as policosanol) and a preparation of chromium and/or chromium salts.

2. Description of the State of Art

According to the National Institute of Health National Cholesterol Education Program (NIH NCEP), approximately 22% of all Americans are affected by Metabolic Syndrome, which is defined by a cluster of symptoms that include abdominal obesity, impaired glucose tolerance, dyslipidemia and raised blood pressure. Metabolic Syndrome is linked to increased risk of heart disease and type 2 diabetes.

Heart Disease

According to the American Heart Association (AHA), about 62 million Americans have some form of cardiovascular disease, which can include high blood pressure, coronary heart disease (heart attack and chest pain), stroke, birth defects of the heart and blood vessels, and congestive heart failure, and close to a million die from such conditions every year. The annual report of the AHA further states that cardiovascular disease kills more Americans than the next 7 causes of death combined, including cancer. Surprisingly, slightly more females, overall, than males have cardiovascular disease. Heart disease accounted for 40% of all deaths in the U.S. in 1999.

According to the National Heart, Lung, and Blood Institute (NHLBI) the higher your blood cholesterol, the greater your risk for developing heart disease and suffering a heart attack. Thus, the primary treatment goal for the prevention of coronary heart disease is to achieve recommended cholesterol levels. However, because high blood cholesterol does not cause any symptoms, many people (more than 50 percent by recent estimates) are either inadequately treated or unaware that their cholesterol level is too high. Considering that 41 million estimated American adults have high cholesterol (according to the AHA), the failure to appreciate high cholesterol's importance places many people at unnecessary risk for developing future heart disease.

Cholesterol is a soft waxy, fat-like substance that is necessary for good health. It is a normal component of most body tissues, especially those of the brain, nervous system, muscle, skin, liver, intestines, and heart. Without cholesterol, our bodies could not function properly. It is needed to form the sex and adrenal hormones, vitamin D and bile (a digestive secretion required for fat digestion).

Cholesterol in the body comes from two major sources. The first is from the liver, which is the body's major cholesterol-producing organ. The second source is from eating animal products such as meat (beef, chicken, fish), egg yolks, cheese and other whole milk products. Because the liver is usually able to make enough cholesterol to satisfy all of our bodily needs, too much dietary cholesterol can lead to high bodily levels of cholesterol. These high levels are undesirable because it is difficult for our bodies to appropriately dispose of excess cholesterol.

Cholesterol, triglycerides, and other lipid molecules are transported through the bloodstream by protein spheres called lipoproteins. Most of the information about the effects of cholesterol and triglyceride actually concerns lipoproteins. Lipoproteins are categorized into five types according to size and density. They can be further defined by whether they carry cholesterol (the two smaller lipoproteins) or triglycerides (the three largest lipoproteins).

Cholesterol-carrying lipoproteins (low-density and high-density lipoproteins) are the lipoproteins commonly referred to as cholesterol. Cholesterol also behaves differently depending on which type of lipoprotein carries it. Low Density Lipoprotein (LDL) transports about 75% of the blood's cholesterol to the body's cells. It is normally harmless. However, if it is exposed to a process called oxidation, it can penetrate and interact dangerously with the walls of the artery, producing a harmful inflammatory response. When LDL collects on arterial walls oxidants are produced and released from the wall membranes. These oxidants tend to bind to and modify the LDL, thereby signaling the immune system that a harmful molecule has appeared. In response to oxidized LDL, the body releases various immune factors aimed at protecting the damaged walls. Unfortunately, in excessive quantities they cause inflammation and promote further injury to the areas they target. White blood cells and other factors gather and form the fatty substance called plaque. Over time the growth of plaque on the artery walls narrow the artery and obstructs the flow of blood. This is referred to as atherosclerosis or “hardening of the arteries”. If the blood flow to the heart is blocked, a heart attack can occur. If the blood flow to the brain is blocked, a stroke can occur. Since LDLs promote atherosclerosis, they are known as “bad cholesterol.” The NHLBI classification of the optimal level of LDL cholesterol is less than 100 milligrams (mg) per deciliter (dL). Borderline high is 130-159 mg/dL, and very high is 190 mg/dL and above. High LDL cholesterol always requires attention. Since the majority of cholesterol is in the form of LDLs, a high blood cholesterol level means high LDL levels and the higher the LDL level, the higher the risk of heart problems.

Lipoprotein(a) (Lp(a)) is a type of LDL cholesterol modified by the addition of an apolipoprotein in the liver. There is a significant association between high levels of Lp(a) and an increased risk of cardiovascular disease. The median level of Lp(a) in the general population is 4 mg/dL. About 20% of the population appears to have increased levels of Lp(a), a purely genetic characteristic, and those in the 90th percentile have an average of 18 mg/dL. Lowering a high level of Lp(a) is difficult. The best means of reduction is to decrease LDL cholesterol as much as possible, since lowering LDL cholesterol substantially decreases the risk associated with elevated Lp(a).

High Density Lipoprotein (HDL) or good cholesterol actually removes cholesterol from the walls of arteries and brings it back to the liver to be safely excreted. It also helps prevent oxidation of LDL. In fact, it appears to have antioxidant properties on its own. People who exercise, don't smoke, and stay at their ideal weight tend to have higher levels of HDLs. HDL cholesterol protects against heart disease. This means that higher numbers of HDL cholesterol are better. A level less than 40 mg/dL is considered low and a major risk factor for the development of coronary artery disease. HDL levels of 60 mg/dL or more help to lower your risk for heart disease.

The remaining three types of lipoproteins, that is, intermediate density lipoproteins (IDL), very low-density lipoproteins (VLDL), and chylomicrons are triglyceride-carrying lipoproteins. Triglycerides are another type of substance closely related to cholesterol. While less is known about triglycerides, in general, there is some evidence to suggest that they are a particularly important cause of coronary artery disease among women and people with other risk factors such as diabetes and obesity. Triglycerides also can raise heart disease risk. Some evidence also suggests that high triglycerides are risk factors for heart disease on their own regardless of cholesterol levels. Levels that are borderline high (150-199 mg/dL) or high (200 mg/dL or more) may require treatment for some people.

According to the new guidelines released in May 2001 by the NHLBI's National Cholesterol Education Program (NCEP), everyone age 20 and older should have their cholesterol and triglyceride levels measured at least once every five years. This blood test is done after a 9- to 12-hour fast and provides information about one's total cholesterol (TC), LDL and HDL cholesterol, and triglycerides. If the total blood cholesterol is 200 milligrams (mg) per deciliter (dL) or more, or if your HDL level is less than 40 mg/dL, a physician should be consulted on ways to lower one's total blood cholesterol.

More recently, experts have begun to examine the individual components of the lipid profile, in addition to the total cholesterol level. While an elevated total cholesterol level is a risk factor, the levels of the various forms of cholesterol which make up the total cholesterol may be a better indication of risk factors. For example, studies indicate that the ratio of LDL cholesterol to HDL cholesterol is more important than individual levels of LDL cholesterol and HDL cholesterol in that the ratio is a more accurate measure of risk of cardiovascular disease. The higher the LDL/HDL ratio, the higher the risk of cardiovascular disease. Ideally, the LDL/HDL ratio should not exceed 4.4. An LDL/HDL ratio in the range of 4.4 to 7.1 is considered to indicate an average risk of cardiovascular disease. A moderate risk ratio is 7.1 to 11, and any ratio above 11 is considered to indicate a high risk of cardiovascular disease.

Evidence has been accumulating in recent years that driving cholesterol even lower than the current guidelines recommend may produce additional benefits. However, researchers have been hesitant to begin prescribing higher dosages of the costly drugs until they had clear evidence it would keep people healthier and reduce their risk of dying.

Lowering blood cholesterol levels is important for everyone, including younger, middle-aged, and older adults, and people with or without heart disease and/or stroke. Lowering blood cholesterol levels that are too high lessens the risk for developing heart disease and reduces the chance of a heart attack or dying of heart disease. This is especially true for people who have already suffered a heart attack. Blood cholesterol levels are affected by many factors, which includes diet, increasing exercise, or medication. This is very important because with every 1 percent reduction in total blood cholesterol, there is about a 2 percent reduction in the risk of heart attack.

When a patient without heart disease is first diagnosed with elevated blood cholesterol, physicians often prescribe a program of diet, exercise, and weight loss to bring levels down. The National Cholesterol Education Program guidelines suggest at least a six-month program of reduced dietary saturated fat and cholesterol, together with physical activity and weight control, as the primary treatment before resorting to drug therapy. Typically, physicians prescribe the Step I/Step II diet devised by the National Institutes of Health, National Heart, Blood and Lung Institute, aimed at lowering LDL cholesterol. The goals of the Step I Diet are to limit cholesterol intake to less than 300 mg per day and fat intake to 30 percent or less of the day's total calories, with only 8 percent to 10 percent of calories from saturated fat. The more aggressive Step II Diet limits cholesterol intake to less than 200 mg per day and fat intake to 30 percent or less of the day's total calories, with less than 7 percent of total calories from saturated fat. Many patients respond well to this diet and end up sufficiently reducing blood cholesterol levels.

People who are on a cholesterol-lowering diet, however, are successful in actually lowering their risk for heart disease only if they also follow a regular aerobic exercise program. Some studies suggest that for the greatest heart protection, it is not the duration of a single exercise session that counts but the total daily amount of energy expended. Therefore, the best way to exercise may be in multiple short bouts of intense exercise. Burning at least 250 calories a day (the equivalent of about 45 minutes of brisk walking or 25 minutes of jogging) seems to confer the greatest protection against coronary artery disease, most likely because it raises HDL levels. Note, however, moderate exercise has little effect on HDL, and it may take up to a year of sustained exercise to make any significant difference on HDL levels.

Aerobic exercise appears to raise HDL levels, open up the blood vessels and, in combination with a healthy diet, may improve blood-clotting factors. Resistance (weight) training offers a complementary benefit to aerobics by reducing LDL levels.

Diabetes

According to the American Diabetes Association (ADA), about 18.2 million Americans, or 6.3% of the population, have diabetes. While an estimated 13 million have been diagnosed, it is estimated that 5.2 million people (or nearly one-third) are unaware that they have the disease. The ADA also reports that diabetes is the sixth leading cause of death in America. Diabetes is likely to be under reported as a cause of death because many decedents with diabetes do not have the disease entered on their death certificate. Instead, one of the major complications related to diabetes is often listed. Heart disease, stroke and high blood pressure are the leading diabetes-related complications.

Diabetes is a group of diseases characterized by high levels of blood glucose resulting from defects in insulin production, insulin action, or both. The primary types of diabetes include type 1 diabetes, type 2 diabetes and gestational diabetes. In all forms of diabetes, high levels of blood glucose increase the risk for diabetes-related complications such as heart disease, stroke, high blood pressure, kidney disease, blindness and nerve damage. The high levels of blood glucose occur because insulin, the hormone essential for regulating the storage and use of glucose and amino acids in the body, is either not produced (type 1 diabetes) or cells become resistant to insulin (type 2 diabetes).

Type 1 diabetes develops when the body's immune system destroys pancreatic beta cells, the only cells in the body that make the hormone insulin that regulates blood glucose. This form of diabetes usually strikes children and young adults, although disease onset can occur at any age. It is estimated that type 1 diabetes accounts for 5% to 10% of all diagnosed cases of diabetes. Risk factors for type 1 diabetes include autoimmune, genetic, and environmental factors.

Type 2 diabetes is the most common form of diabetes. It is estimated that type 2 diabetes accounts for about 90% to 95% of all diagnosed cases of diabetes. Type 2 diabetes usually begins as insulin resistance, a disorder in which the cells do not use insulin properly. As the need for insulin rises, the pancreas gradually loses its ability to produce insulin. Type 2 diabetes is associated with older age, obesity, family history of diabetes, prior history of gestational diabetes, impaired glucose tolerance, physical inactivity, and race/ethnicity.

Gestational diabetes is a form of glucose intolerance that is diagnosed in some women during pregnancy. During pregnancy, gestational diabetes requires treatment to normalize maternal blood glucose levels to avoid complications in the infant. After pregnancy, 5% to 10% of women with gestational diabetes are found to have type 2 diabetes. Women who have had gestational diabetes have a 20% to 50% chance of developing diabetes in the next 5-10 years. Risk factors for gestational diabetes include race/ethnicity, obesity and family history of diabetes.

Type 2 diabetes generally begins with insulin resistance. Insulin resistance occurs when the body fails to respond properly to the insulin it already produces. Insulin resistance can also occur in people who have type 1 diabetes, especially if they are overweight. Many people with insulin resistance and high blood glucose have excess weight around the waist, high LDL blood cholesterol levels, low HDL cholesterol levels, high levels of triglycerides, and high blood pressure, all conditions that also put the heart at risk. This combination of problems is referred to as the metabolic syndrome.

According to the NIH, anyone 45 years or older should consider getting tested for diabetes. Those who are overweight and aged 45 or older, are strongly urged to get tested. Furthermore, one should consider getting tested if younger than 45, overweight, and exhibiting one or more additional risk factors.

A fasting glucose test measures blood glucose after fasting overnight. Fasting glucose levels of 100 to 125 mg/dL are above normal but not high enough to be called diabetes. This condition is called pre-diabetes or impaired fasting glucose, and it suggests insulin resistance. Impaired fasting glucose is considered a pre-diabetic state. A fasting glucose test result of 126 or higher, if confirmed on a repeat test, indicates diabetes.

A glucose tolerance test measures blood glucose after an overnight fast and 2 hours after drinking a sugar solution. If blood glucose falls between 140 and 199 mg/dL 2 hours after drinking the liquid, glucose tolerance is above normal but not high enough for diabetes. This condition, also a form of pre-diabetes, is called impaired glucose tolerance and, like impaired fasting glucose, points toward a history of insulin resistance and a risk for developing diabetes. A glucose tolerance level of 200 or higher, if confirmed, indicates the presence of diabetes.

When a person is first diagnosed with insulin resistance, physicians often prescribe a program of diet, exercise, and weight loss to help the body relearn to use insulin normally. Achieving a healthy cholesterol level is also considered to be significant. Although energy restriction and increased exercise improve insulin resistance, they long-term success of these methods is poor. Therefore, strategies to improve insulin resistance by pharmacological means or by nutritional supplementation represents a very attractive approach.

Therapeutic Treatment

In many cases, a real change in diet along with more physical activity may not be enough to improve glucose levels, cholesterol levels and lean body mass. Therapeutic treatment should be considered for patients who, in spite of dietary changes, regular physical activity and weight loss, need further treatment for elevated blood glucose, LDL cholesterol and triglyceride levels. Perhaps a genetic predisposition exits. In these cases, physicians often prescribe drugs. The National Cholesterol Education Program estimates that as many as 9 million Americans take some form of cholesterol-lowering drug therapy. Currently, there are prescription medications available that lower cholesterol, such as the Niacins, Statins, Fibrates, and Resins. These prescription medications, however, are linked to various forms of severe side effects including liver and kidney failure and cancer.

As an alternative to prescription medications, nutritional supplements may also be used to lower total blood cholesterol levels. Examples of nutritional supplements that appear to be effective are: Coenzyme Q10 (CoQ10); L-carnitine; garlic; digestive enzymes, such as lipase and amylase; probiotics or “friendly bacteria” such as L. Acidophilus; Milk Thistle (Silybum marianum); herb tea; pantethine and pantothenic acid; chromium piccolinate, and policosanol.

The U.S. Department of Agriculture and the Department of Health and Human Services recommend nutritional supplements for a variety of reasons. For one, the cost of supplements is significantly lower than the costs of medications. It is recommended that most adults use nutritional supplements to reduce their risk of chronic diseases including heart disease and diabetes.

Recent studies have shown that chromium, in the form of chromium picolinate, can improve insulin metabolism and has lipid-lowering activity. There is evidence demonstrating the benefits of chromium picolinate with respect to diabetes, obesity and hypercholsterolemia. Research has demonstrated that a lack of chromium can cause insulin resistance in both human clinical and isolated tissues studies.

It is speculated that chromium picolinate reduces insulin resistance by stimulating the activity of insulin, thus significantly aiding the body's glucose and fat metabolism, managing the breakdown of glucose and fat. The exact mechanisms by which chromium improves insulin efficiency are currently unclear. It has been suggested that chromium somehow works to increase sensitivity of insulin receptors and facilitates the absorption of glucose and protein and the metabolism of fat (Krzanowski, J. of the Florida Med. Ass'n, 83(1):29-31). Chromium theoretically helps insulin transport glucose and amino acid molecules inside the cells for energy production and tissue syntheses. Chromium binds insulin to a special receptor site on cellular membranes, and aids insulin absorption. Because chromium increases tissue sensitivity to insulin, it decreases insulin levels in the body, and subsequently decreases fat storage in the tissues.

Because chromium augments the action of insulin, the body's primary anabolic hormone, it is believed that chromium may facilitate muscular development. Chromium increases insulin's ability to transport glucose and amino acid molecules from the blood into muscle cells. Because amino acids are the building blocks of protein, it is theorized that this increased transport of amino acids into muscle cells may increase muscle protein and boost lean muscle mass.

Several different preparations of chromium are available including chromium picolinate, chromium chloride, chromium nicotinate and high-chromium yeast. Nicotinate and picolinate seem more easily absorbed than others. Studies indicate that the most beneficial and bio-available form is chromium picolinate, and studies using other forms of chromium have produced varying effects. Chromium picolinate combines chromium with picolinic acid, a compound which helps the body better absorb and process minerals. In various clinical trials, chromium picolinate has distinguished itself as the best form of chromium.

Chromium picolinate, however, has been met with safety concerns regarding the adverse side effects. Recent laboratory studies have found that chromium picolinate could damage genetic material in animal cells, which suggests that it might cause cancer. Chromium picolinate reacts with Vitamin C and other antioxidants in the cells to produce a reduced from of chromium, capable of causing mutations in DNA.

Moreover, the chromium compound eventually disassociates from the piccolonate compound and has been shown to have adverse effects individually. Many of these adverse effects stem from the fact that chromium, like many other minerals, does not wash out of the body. Chromium tends to accumulate in the body, where it may cause of worsen kidney or liver damage.

Chromium also appears to affect the levels of neurotransmitters in the body. Therefore, individuals who have anxiety, depression or psychiatric conditions have been advised not to take chromium. In some cases, individuals have reported headache, insomnia and mood changes. In others, progressively worse cognitive, perceptual and motor changes have occurred. Other adverse side effects include anemia and red, itchy, scaly rashes.

Moreover, while the effects of chromium on insulin and glucose levels have been investigated, the effects of chromium on heart disease, a major concern for those with diabetes and Metabolic Syndrome, remain unclear. There is some evidence suggesting that chromium may reduce triglyceride levels. However, little is known regarding the effects of chromium on raising HDL and lowering LDL cholesterol levels.

A mixture of high purity, high molecular weight straight chain aliphatic alcohols (collectively referred to herein as policosanol) has garnered much interest in recent years as a natural supplement for treating heart disease, most notably due to its cholesterol-lowering effects, Gouni-Berthold I., et al., Am Heart J, 143(2):356-365 (2002). The main constituents of policosanol are tetracosanol, hexacosanol, octacosanol, and triacontanol, while eicosanol, docosanol, heptacosanol, nonacosanol, dotriacontanol, tetratriacontanol, and hexatriacontanol make up the remaining minor constituents of the straight chain aliphatic alcohols. There is a significant body of evidence demonstrating the benefits of policosanol with respect to cardiovascular disease. In the mid to late nineties, one research group proposed that policosanol was able to reduce endothelial damage by inhibiting the production of foam cells (Noa M., et al., J Pharm Pharmacol, 48(3):306-309 (1996); Noa M., et al., J Pharm Pharmacol, 49(10):999-1002 (1997). Foam cells are macrophages that can migrate into the endothelium of the blood vessels and contribute to atherosclerotic plaque formation (Physicians' Desk Reference. 50 ed. Montvale, N.J.: Medical Economics Company; 2002.). Other researchers believe policosanol has a modulating effect on HMG-CoA reductase, the rate-controlling enzyme in cholesterol biosynthesis, but the precise mechanism remains unclear (Menendez R., et al., Biol Res, 27(3-4): 199-203 (1994); Menendez R., et al., Biol Res, 29(2):253-257 (1996); and Menendez R., et al., Arch Med Res, 32(1):8-12 (2001). Still, other investigators believe policosanol may inhibit cholesterol synthesis in the liver at a step before mevalonate production, but total inhibition of the HMG-CoA reductase is doubtful (Gouni-Berthold I., et al., Am Heart J, 143(2):356-365 (2002). More recent work suggests policosanol inhibits LDL cholesterol oxidation (Menendez R., et al., Can J Physiol Pharmacol, 80(1):13-21 (2002); Menendez R., et al., Br J Clin Pharmacol, 50(3):255-262 (2000). This was revealed when markers of peroxidation, such as thiobarbituric acid reactive substances (TBARS), and malondialdehyde (MDA) were lower in the cultures treated with policosanol. Oxidation of LDL cholesterol has been linked to heart disease and was the recent cover story in Scientific American magazine (Physicians' Desk Reference. 50 ed. Montvale, N.J.: Medical Economics Company; 2002). Bi-products of LDL oxidation are bioactive, and secrete inflammatory cytokines, growth factors and cell surface adhesion molecules. In response to these oxidative bi-products, smooth muscle cells proliferate in the wall of the artery, resulting in the narrowing of the lumen and eventual blockage. Oxidized LDL cholesterol can also inhibit the production of prostacyclin and nitric oxide, which act as vasodilators and inhibitors of platelet aggregation.

While there are no known side effects related to the use of policosanol and the percentage decrease in the reduction of total cholesterol as well as total LDL-cholesterol is statistically significant, it is not as significant as the reduction that occurs as a result of administering the prescription medications discussed previously.

It would be advantageous to provide a unique policosanol-containing formulation which allows individuals to significantly lower and maintain healthy insulin and cholesterol levels in the blood, while not exposing the individual to the same deleterious side effect that result from long term use of prescription medications or chromium supplements.

### SUMMARY OF THE INVENTION

The present invention provides a therapeutic composition for reducing serum cholesterol levels, LDL-cholesterol levels, LDL/HDL ratios, blood glucose levels and for increasing lean body mass in humans and animals, and a method for reducing serum cholesterol levels, LDL-cholesterol levels, LDL/HDL ratios, blood glucose levels and for increasing lean body mass in humans and animals by administering the composition of the present invention. The composition of the present invention comprises a mixture of high purity, high molecular weight straight chain primary aliphatic alcohols and chromium and/or chromium salts, wherein the composition comprises from about 1% to about 90% by weight policosanol and from about 5% to about 75% by weight of chromium and/or chromium salts. The composition further comprises from 0% to about 65% by weight of pharmaceutically acceptable formulation aids, such as diluents, stabilizers, binders, buffers, lubricants, coating agents, preservatives, emulsifiers and suspension agents.

In one embodiment of this aspect of the invention, the policosanol comprises at least one high molecular weight straight chain primary aliphatic alcohol selected from 20 to 36 carbon atoms, and the composition is further characterized by a combination policosanol and chromium and/or chromium salts in a quantitative ratio from 100:1 to 0.01:1 by weight.

In another embodiment of the composition of the present invention, the policosanol comprises 1-tetracosanol, 1-hexacosanol, 1-heptacosanol, 1-octacosanol, 1-triacontanol, 1-dotriacontanol and 1-tetratriacontanol; and a chromium salt, and the composition is further characterized by a combination of policosanol and chromium salt in a quantitative ratio from 10:1 to 0.10:1 by weight.

In yet another embodiment, the composition of the present invention comprises policosanol having the following quantitative composition:

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 0-30% |
| 1-hexacosanol (C₂₆) | 5-30% |
| 1-heptacosanol (C₂₇) | 0-5% |
| 1-octacosanol (C₂₈) | 5-80% |
| 1-nonacosanol (C₂₉) | 0-5% |
| 1-triacontanol (C₃₀) | 5-40% |
| 1-dotriacontanol (C₃₂) | 1-25% |
| 1-tetratriacontanol (C₃₄) | 0-7% |
| 1-hexatriacontanol (C₃₆) | 0-5% |

and chromium and/or chromium salts which may be selected from the group consisting of chromium picolinate, chromium chloride, chromium nicotinate, chromium polynicotinate, chromium acetate, trivalent chromium, and high-chromium yeast; and the composition is further characterized by a combination of policosanol and chromium and/or chromium salts in a quantitative ratio from 3:1 to 0.10:1 by weight.

In still another aspect, the present invention relates to a method for treating or preventing metabolic syndrome, hypercholesterolemia related diseases, and hypoglycemia related diseases, which comprises administering a pharmaceutically effective amount of a composition comprising policosanol and chromium and/or chromium salts to a mammal, e.g., a human.

In yet another aspect, the present invention relates to a method for reducing total cholesterol and LDL-cholesterol levels, while also reducing blood glucose levels and increasing lean body mass, which comprises administering a pharmaceutically effective amount of a composition comprising policosanol and chromium and/or chromium salts to a mammal, e.g., a human, in need thereof.

In still yet another aspect, the present invention relates to a method of using a composition comprising policosanol and chromium and/or chromium salts which comprises administering said composition to reduce and/or prevent metabolic syndrome, hypercholesterolemia and hypoglycemia related diseases, total cholesterol, LDL-cholesterol, LDL/HDL ratio, triglycerides, coronary heart disease (heart attacks and strokes), inflammation, deep-vein thrombosis, immunoregulatory diseases, cardiovascular diseases, obesity, insulin resistance, dyslipidemia, raised blood pressure, fatigue, premenstrual syndrome, anxiety, depression and/or neurodegenerative disorders, and/or raise HDL cholesterol and/or lean body mass, in an individual in need thereof. The daily dosage is established between 1 to 100 mg of policosanol (preferably 3 to 20 mg) and 0.05-2,000 mcg of chromium and/or chromium salts per day and is intended for ingestion in any type or form of foodstuff, capsule, tablet or liquid form.

The present invention further contemplates providing kits having one or more containers comprising the therapeutic composition of the present invention and a suitable excipient as described herein and a set of instructions, generally written instructions although electronic storage media (e.g., magnetic diskette or optical disk) containing instructions are also acceptable, relating to the use and dosage of the therapeutic composition of the present invention for the intended treatment. The instructions included with the kit generally include information as to dosage, dosing schedule, and route of administration for the intended treatment. The containers of the therapeutic composition of the present invention may be unit doses, bulk packages (e.g., multi-dose packages) or sub-unit doses.

### DETAILED DESCRIPTION OF THE INVENTION

The composition of the present invention comprises a mixture of high purity, high molecular weight straight chain primary aliphatic alcohols (referred collectively herein to as policosanol) and chromium and/or chromium salts as the primary therapeutic agents to be administered for the purpose of reducing and/or preventing metabolic syndrome, hypercholesterolemia and hypoglycemia related diseases, total cholesterol, LDL-cholesterol, LDL/HDL ratio, triglycerides, coronary heart disease (heart attacks and strokes), inflammation, deep-vein thrombosis, immunoregulatory diseases, cardiovascular diseases, obesity, insulin resistance, dyslipidemia, raised blood pressure, fatigue, premenstrual syndrome, anxiety, depression and/or neurodegenerative disorders, and/or raise HDL cholesterol and/or lean body mass, in an individual in need thereof.

Policosanol may be extracted and purified from a wide array of starting materials, such as, but not limited to, natural waxes, such as, but not limited to, beeswax, carnauba wax, and candellia wax; bee pollen; oils, such as, but not limited to, peanut oil, sesame oil, cod liver oil, rice bran oil, oat oil, and rosemary needles oil; and powders, such as, but not limited to rice bran, containing primarily natural esters of aliphatic alcohols with carboxylic acids. Consequently, the quantitative compositions of policosanol can vary depending on the extraction process and starting materials that are used in its production. In general, it is possible to obtain policosanol having the following quantitative composition:

TABLE I

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 0-30% |
| 1-hexacosanol (C₂₆) | 5-30% |
| 1-heptacosanol (C₂₇) | 0-5% |
| 1-octacosanol (C₂₈) | 5-80% |
| 1-nonacosanol (C₂₉) | 0-5% |
| 1-triacontanol (C₃₀) | 5-40% |
| 1-dotriacontanol (C₃₂) | 1-25% |
| 1-tetratriacontanol (C₃₄) | 0-7% |
| 1-hexatriacontanol (C₃₆) | 0-5% |

U.S. Pat. Nos. 5,663,156; 5,856,316; 6,197,832; 6,225,354; and 6,596,776, all of which are incorporated herein by reference disclose policosanol compositions that are specific to the starting material and extraction processes used. It should be noted that while any commercially available policosanol or any of the policosanols disclosed in the above-referenced patents are suitable for use in the present invention, for purposes of the remainder of this discussion the policosanol and methodologies disclosed in U.S. Pat. No. 6,596,776 will be referenced. Specifically, the policosanol used in the present invention is obtained from beeswax and has the formulation set forth below in Table II.

TABLE II

| | Proportion in |
|---|---|
| Components | the mixture |
| 1-eicosanol (C₂₀) | 0-5% |
| 1-docosanol (C₂₂) | 0-5% |
| 1-tetracosanol (C₂₄) | 13-28% |
| 1-hexacosanol (C₂₆) | 5-30% |
| 1-heptacosanol (C₂₇) | 0-5% |
| 1-octacosanol (C₂₈) | 15-25% |
| 1-triacontanol (C₃₀) | 25-40% |
| 1-dotriacontanol (C₃₂) | 5-15% |
| 1-tetratriacontanol (C₃₄) | 0-5% |

The process used to isolate the policosanol, described in Table II above, is incorporated herein by reference and is briefly described as follows. Beeswax is initially subjected to a homogenous phase saponification step after which the saponified beeswax is dried and ground to a particle mesh size of 100-500 microns. Alternatively, unsaponified beeswax, of varying purity, may be used as the starting material and is initially dried and ground to a particle mesh size of 100-200 microns. The particles of saponified or unsaponified beeswax are placed into a conventional solid-liquid extractor and a hot organic solvent is introduced and contacted with the beeswax particles. The suspension is mixed and then hot-filtered to remove any solids.

The resulting extract is then maintained within the temperature range of 2° C.-10° C. causing the aliphatic alcohols to solidify and form a suspension. The suspension is filtered and the first solids are recovered and air dried. The dried solids obtained after drying are then sent to a purifier where they are contacted with and dissolved in a second hot solvent and hot-filtered. This solution is then cooled and the second solids collected and dried by vacuum. The dried solids obtained from the second purification step are contacted with another hot organic solvent, which dissolves the solids. This solution is hot-filtered and chilled, and the third solids collected, dried, and powdered to become the final product disclosed in Table II above.

After the particles are dried, they are then ready to be combined with chromium and/or chromium salts thereby forming the therapeutic composition of the present invention which is then formulated into a conventional pharmaceutical formulation such as tablets, capsules, etc., for administration.

As discussed previously, chromium and/or chromium salts and policosanol lower serum cholesterol by two independent mechanisms of action. However, both compounds together are expected to have a synergistic effect on lowering serum cholesterol and blood glucose, while increasing lean body mass. As previously mentioned, the mode of action of the chromium and/or chromium salts is to improve insulin activity, thus aiding glucose and fat metabolism as well as increasing protein synthesis and the production of seratonin. The consequence of this action is trifold. First, the improved fat metabolism regulates the fat production process. Thus, chromium and/or chromium salts reduce serum cholesterol by lowering blood lipids, serum cholesterol and triglyceride levels. Second, the improved glucose metabolism facilitates the conversion of blood glucose to glycogen by insulin. Thus, chromium and/or chromium salts reduce insulin resistance. Third, the increased protein synthesis and production of seratonin, a hormone that reduces appetite, work to reduce fat while increasing lean body mass. Policosanol, on the other hand, acts directly on the cholesterol synthesis pathway itself, thereby inhibiting the bio-synthesis of cholesterol from saturated fat. Furthermore, the side effects of chromium and/or chromium salts are well known and usually limit usage. As described above, high doses cause kidney and liver failure and other uncomfortable side effects that adversely affect patient compliance. In addition, certain forms of chromium can also cause cancer. Consequently both compounds together, policosanol and chromium and/or chromium salts, will have a synergistic effect on lowering serum cholesterol levels and blood glucose levels and increase lean muscle mass. Thus, the combination of both policosanol and chromium and/or chromium salts into a single composition is expected to provide a more effective treatment for elevated serum cholesterol and blood glucose than would be expected from the additive effect of both components. Furthermore, it is expected that the composition of the present invention will contain a significant decrease in the recommended daily dosage of the chromium and/or chromium salts constituent thus decreasing the side effects associated with the use of chromium and/or chromium salts while simultaneously achieving an effective treatment for elevated scram cholesterol, LDL-cholesterol, and blood glucose.

The chromium currently available for use in the present invention and the associated recommended daily dosage include non-prescription chromium. The chromium salts include, chromium picolinate (chromium, tris (2-pyridinecarboxylato-N(1), O(2))-(9CI)), chromium chloride, chromium nicotinate, chromium polynicotinate, chromium acetate, trivalent chromium, high-chromium yeast, chromium 2-pyridine-carboxylate, chromium tripicolinate, 2-pyridinecarboxylic acid-chromium salt, and tris(picolinato)chromium. The dose of these medicines will be variable for different patients and dose levels can be determined as is normally employed in the art, for example, as indicated in the Physician's Desk Reference and The Merck Index (Twelfth Edition), the contents of both of which are incorporated herein by reference.

Policosanol and Chromium and/or Chromium Salts Formulations of the Present Invention

The formulations of the present invention comprise compositions made by combining policosanol with chromium and/or chromium salts. Such compositions can comprise policosanol with chromium and/or chromium salts in a quantitative ratio from about 100:1 to about 0.01:1 by weight, to from about 10:1 to about 0.10:1 by weight, e.g., from about 3:1 to about 0.33:1 by weight, and more typically from about 2:1 to about 0.5:1. Compositions of the present invention may further contain 1:1 weight ratios of policosanol with chromium and/or chromium salts.

Policosanol is extremely well tolerated. In animal toxicity studies, doses up to 500 mg/kg/day, a dose that is 1500 times the normal human dose of 20 mg/day have shown no negative effects on carcinogenesis, reproduction, growth, and development. Total doses of policosanol according to the present invention range from 1 mg to 100 mg per day, in another embodiment it is contemplated that 5 mg to 40 mg per day is used and in yet another embodiment it is contemplated that the dose would be in the range of 10 to 20 mg per day.

A wide variety of chromium and/or chromium salt preparations are available from different manufacturers, each having unique bioavailability, pharmokinetic, and safety profiles. In general, lower doses of chromium and/or chromium salts are contemplated for use because they maintain beneficial lipid and insulin effects while minimizing adverse side affects. The total dose for this component of the composition of the present invention can range from about 0.05-2,000 mcg/day, or any other dose, depending upon the specific chromium and/or chromium salts employed. According to the present invention, one of the synergistic effects of the active compounds that make up the composition of the present invention is the ability to achieve the same end results that can possibly be achieved with the use of the chromium and/or chromium salts alone while significantly decreasing the side effects associated with the use of chromium and/or chromium salts. Consequently, while the recommended daily dosage of each chromium and/or chromium salt can be followed and combined with policosanol to form the composition of the present invention, it is preferable to use a smaller dose than recommended. With respect to chromium and/or chromium salts, it is contemplated that a useful dose is in the range of 0.05-2,000 mcg/day, in another embodiment the useful dose is in the range of 0.1-1,000 mcg/day, in another embodiment the useful dose is in the range of 0.2-70 mcg/day for chromium, while the useful dose for a chromium salt is in the range of 100-1,000 mcg/day. Other dosing ranges may be further determined by one skilled in the art as indicated in the Physician's Desk Reference and The Merck Index (Twelfth Edition).

The compositions of the present invention can be taken in amounts sufficient to provide the desired dosages discussed above. The formulation can be taken once or more times a day.

The pharmaceutical formulations of the present invention can contain as active ingredients from about 0.5 to about 95.0% wt of policosanol and chromium and/or chromium salts. This dosage is obtained by mixing the policosanol and chromium and/or chromium salts with different excipients such as agglutinants, disintegrators, lubricants, sliders or just fillers. These excipients include lactose, corn starch, saccharose, magnesium stearate, microcrystalline cellulose, sodium croscarmellose gelatin, cellulose acetophtalate, titanium dioxide, special talc for tablets and polyethylene glycol.

The pharmaceutical composition of the present invention may be administered to humans and animals. The daily dosage of this composition to be used for the reduction and/or prevention of metabolic syndrome, hypercholesterolemia and hypoglycemia related diseases, total cholesterol, LDL-cholesterol, LDL/HDL ratio, triglycerides, coronary heart disease (heart attacks and strokes), inflammation, deep-vein thrombosis, immunoregulatory diseases, cardiovascular diseases, obesity, insulin resistance, dyslipidemia, raised blood pressure, fatigue, premenstrual syndrome, anxiety, depression and/or neurodegenerative disorders, and/or raise HDL cholesterol and/or lean body mass, is established between 1 to 100 mg/day for the policosanol substituent and 0.05-2,000 mcg/day for the chromium and/or chromium salt substituent depending on which chromium and/or chromium salt is present and is intended for administration in a variety of ways discussed in further detail below. It is also helpful for the patient to take 162 mg to 325 mg of aspirin 30 minutes before administration of the composition of the present invention.

The therapeutic compositions of the present invention comprise chromium and/or chromium salts and policosanol. The policosanol used in the present invention can be derived from any suitable source, each source being associated with a policosanol of particular characteristics, usually in terms of the relative proportions of its primary aliphatic alcohol components and the composition of the present invention is further characterized by a combination of policosanol and chromium and/or chromium salts in a quantitative ratio from 10:1 to 0.01:1 by weight. The therapeutic composition of the present invention may further comprise aspirin in the range of 162-325 mg.

The therapeutic composition of the present invention may be packaged in any convenient, appropriate packaging.

As will be appreciated by one knowledgeable in the art, the therapeutic composition of the present invention may be combined or used in combination with other treatments known in the art.

The compositions of the invention may be in a form suitable for oral use (for example, as tablets, lozenges, hard or soft capsules, aqueous or oily suspensions, emulsions, dispersible powders or granules, syrups or elixirs), for topical use (for example, as creams, ointments, gels, or aqueous or oily solutions or suspensions), for administration by inhalation (for example, as a finely divided powder or a liquid aerosol), for administration by insufflation (for example, as a finely divided powder) or for parenteral administration (for example, as a sterile aqueous or oily solution for intravenous, subcutaneous, or intramuscular dosing or as a suppository for rectal dosing). For example, compositions intended for oral use may contain, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents, such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example, sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The amount of the active ingredients comprising the composition of this invention that is combined with one or more excipients to produce a single dosage form will necessarily vary depending upon the host treated and the particular route of administration. For example, a formulation intended for oral administration to humans may contain, for example, from 0.05-2,000 mcg of active agent compounded with an appropriate and convenient amount of excipients which may vary from about 5 to about 95 percent by weight of the total composition. Dosage unit forms will generally contain about 0.05-2,000 mcg of an active ingredient. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

In order to use the formulation of policosanol and chromium and/or chromium salts for the therapeutic treatment (including prophylactic treatment) of mammals including humans according to the methods of this invention, it is normally formulated in accordance with standard pharmaceutical practice as a pharmaceutical composition as discussed above. According to this aspect of the invention there is provided a pharmaceutical composition comprising policosanol and chromium and/or chromium salts in association with a pharmaceutically acceptable diluent or carrier, wherein the policosanol and chromium and/or chromium salts are present in an amount for effectively treating or preventing metabolic syndrome, hypercholesterolemia and hypoglycemia related diseases, total cholesterol, LDL-cholesterol, LDL/HDL ratio, triglycerides, coronary heart disease (heart attacks and strokes), inflammation, deep-vein thrombosis, immunoregulatory diseases, cardiovascular diseases, obesity, insulin resistance, dyslipidemia, raised blood pressure, fatigue, premenstrual syndrome, anxiety, depression and/or neurodegenerative disorders, and/or raise HDL cholesterol and/or lean body mass, in an individual in need thereof.

The composition of the present invention can be administered to a patient by any available and effective delivery system including, but not limited to, parenteral, transdermal, intranasal, sublingual, transmucosal, intra-arterial, or intradermal modes of administration in dosage unit formulations containing conventional nontoxic pharmaceutically acceptable carriers, adjuvants, and vehicles as desired, such as a depot or a controlled release formulation.

For example, a pharmaceutically acceptable formulation of the composition of the present invention may be formulated for parenteral administration, e.g., for intravenous, subcutaneous, or intramuscular injection. For an injectable formulation, a dose of the composition of the present invention may be combined with a sterile aqueous solution which is preferably isotonic with the blood of the patient. Such a formulation may be prepared by dissolving a solid active ingredient in water containing physiologically-compatible substances such as sodium chloride, glycine, and the like, and having a buffered pH compatible with physiological conditions so as to produce an aqueous solution, and then rendering the solution sterile by methods known in the art. The formulations may be present in unit or multi-dose containers, such as sealed ampules or vials. The formulation may be delivered by any mode of injection, including, without limitation, epifascial, intracutaneous, intramuscular, intravascular, intravenous, parenchymatous, subcutaneous, oral or nasal preparations (see, for example, U.S. Pat. No. 5,958,877, which is specifically incorporated herein by reference).

Controlled/Extended/Sustained/Prolonged Release Administration

Another aspect of this invention provides methods of treating metabolic syndrome, hypercholesterolemia and hypoglycemia related diseases, total cholesterol, LDL-cholesterol, LDL/HDL ratio, triglycerides, coronary heart disease (heart attacks and strokes), inflammation, deep-vein thrombosis, immunoregulatory diseases, cardiovascular diseases, obesity, insulin resistance, dyslipidemia, raised blood pressure, fatigue, premenstrual syndrome, anxiety, depression and/or neurodegenerative disorders by delivering the composition of the present invention to a patient as a controlled release formulation. As used herein, the terms “controlled”, “extended”, “sustained” or “prolonged” release of the composition of the present invention will collectively be referred to as “controlled release” and includes continuous or discontinuous, linear or non-linear release of the composition of the present invention. There are many advantages for a controlled release formulation of the composition of the present invention. Among these are to effectively suppress cholesterol synthesis during a period when the patient would not be readily able or willing to periodically ingest the composition of the present invention. The composition of the present invention is preferably administered in a single dose. The single dose of composition of the present invention preferably is administered via ingestion of one or more controlled release unit dosage forms so that effective chromium and/or chromium salts and policosanol levels are maintained throughout the critical time periods, i.e., during the peak periods of serum lipid/lipid component biosynthesis and blood glucose biosynthesis.

1. Tablets

A useful controlled release tablet is disclosed in U.S. Pat. No. 5,126,145, which is incorporated by reference herein. This tablet comprises, in admixture, about 5-30% high viscosity hydroxypropyl methyl cellulose, about 2-15% of a water-soluble pharmaceutical binder, about 2-20% of a hydrophobic component such as a waxy material, e.g., a fatty acid, and about 30-90% active ingredient.

More specifically, one such useful controlled release tablet comprises: (a) about 5-20 percent by weight hydroxypropyl methylcellulose having a viscosity of about 10,000 CPS or greater, a substitution rate for the methoxyl group of about 7-30% and a substitution rate for the hydroxypropoxyl group of about 7-20%; (b) about 2-8 percent hydroxypropyl methylcellulose having a viscosity of less than about 100, CPS methyl cellulose, or polyvinyl pyrollidone; (c) about 5-15 percent by weight hydrogenated vegetable oil or stearic acid; and (d) about 30-90% active ingredient.

High viscosity water-soluble 2-hydroxypropyl methyl cellulose (HPMC) is particularly preferred for use in the present tablets and in the controlled-release tablet coating due to its sustaining properties with respect to policosanol and chromium and/or chromium salt release. A particularly preferred high viscosity HMPC has a nominal viscosity, two percent solution, of about 100,000 CPS, methoxyl content of about 19-24, a hydroxypropyl content of about 7-12 percent, and a particle size where at least 90% passes through a USS 100 mesh screen. (Methocel® K100MCR). Low viscosity HPMC is preferred as the binder component of the tablet. A particularly preferred low viscosity HPMC has a methoxyl content of about 20-30%, a hydroxylpropyl content of about 7-12 percent, and a particle size where 100% will pass through a USS No. 30 mesh screen and 99% will pass through a USS 40 mesh screen (Methocel® EIS). In some cases, a portion of the high viscosity HPMC can be replaced by a medium viscosity HPMC, i.e., of about 2000-8,000 cps.

The viscosities reported herein are measured in centipoises (cps or cP), as measured in a 2% by weight aqueous solution of the cellulose either at 20° C. using a rotational viscometer. A “high viscosity” cellulose ether possesses a viscosity of at least about 10,000 cps i.e., about 50,000-100,000 cps. A low-viscosity cellulose ether possesses a viscosity of less than about 100 cps, i.e., about 10-100 cps.

“Water soluble” for purposes of this application means that two grams of powdered cellulose ether can be dispersed by stirring into 100 grams of water at a temperature between 0° C.-100° C. to provide a substantially clear, stable aqueous composition or dispersion (when the dispersion is brought to 20° C.).

Useful hydrophobic components include natural and synthetic waxes such as beeswax, carnauba wax, paraffin, spermaceti, as well as synthetic waxes, hydrogenated vegetable oils, fatty acids, fatty alcohols and the like.

The controlled release policosanol and chromium and/or chromium salt tablets preferably can be formulated to contain 10 mg, 20 mg or 40 mg of policosanol and 0.05-2,000 mcg of chromium and/or chromium salts depending on the particular chromium and/or chromium salts used, and are ingested orally.

Preferably, these tablets will release about 10-35 wt-% of the total policosanol and chromium and/or chromium salts within about 2 hours in an in vitro dissolution test, and about 40-70 wt-% of the total policosanol and chromium and/or chromium salts in eight hours.

These controlled released tablets can also be coated so as to further prolong the release of the chromium and/or chromium salts into the gastrointestinal tract, or to prevent its release into the stomach, in order to maintain consistent levels of chromium and/or chromium salts during critical time periods.

For example, coatings comprising a major portion of a polymeric material having a high degree of swelling on contact with water or other aqueous liquids can be used to further prolong the release of the chromium and/or chromium salts from the tablets core. Such polymers include, inter alia, cross-linked sodium carboxymethylcellulose (Acdisol-FMC), cross-linked hydroxypropylcellulose, hydroxymethylpropylcellulose, e.g., Methocel® K15M, Dow Chem. Co., carboxymethylamide, potassium methylacrylate divinylbenzene copolymer, polymethyl methacrylate, cross-linked polyvinylpyrrolidine, high molecular weight polyvinylalcohol, and the like. Hydroxypropylmethyl cellulose is available in a variety of molecular weights/viscosity grades from Dow Chemical Co. under the Methocel® designation. See also, Alderman (U.S. Pat. No. 4,704,285). These polymers may be dissolved in suitable volatile solvents, along with dyes, lubricants, flavorings and the like, and coated onto the prolonged release tablets, e.g., in amounts equal to 0.1-5% of the total tablet weight, by methods well known to the art. For example, see Remington's Pharmaceutical Sciences, A. Osol, ed., Mack Publishing Co., Easton, Pa. (16th ed. 1980) at pages 1585-1593.

Enteric coatings can also be provided to the prolonged release tablets to prevent release of the chromium and/or chromium salts until the tablet reaches the intestinal tract. Such coatings comprise mixtures of fats and fatty acids, shellac and shellac derivatives and the cellulose acid phthlates, e.g., those having a free carboxyl consent of 9-15%. See, Remington's at page 1590, and Zeitova et al. (U.S. Pat. No. 4,432,966), for descriptions of suitable enteric coating compositions.

2. Films

This invention further provides a prophylaxis for or method of treating a patient following a surgical procedure comprising administering biodegradable, biocompatible polymeric film comprising chromium and/or chromium salts and policosanol to a patient. The polymeric films are thin compared to their length and breadth. The films typically have a uniform selected thickness between about 60 micrometers and about 5 mm. Films of between about 600 micrometers and 1 mm and between about 1 mm and about 5 mm thick, as well as films between about 60 micrometers and about 1000 micrometers; and between about 60 and about 300 micrometers are useful in the manufacture of therapeutic implants for insertion into a patient's body. The films can be administered to the patient in a manner similar to methods used in adhesion surgeries. For example, a policosanol and chromium and/or chromium salts film formulation can be sprayed or dropped onto a tissue site during surgery, or a formed film can be placed over the selected tissue site.

Either biodegradable or nonbiodegradable polymers may be used to fabricate implants in which the chromium and/or chromium salts and policosanol is uniformly distributed throughout the polymer matrix. A number of suitable biodegradable polymers for use in making the biodegradable films of this invention are known to the art, including polyanhydrides and aliphatic polyesters, preferably polylactic acid (PLA), polyglycolic acid (PGA) and mixtures and copolymers thereof, more preferably 50:50 copolymers of PLA:PGA and most preferably 75:25 copolymers of PLA:PGA. Single enantiomers of PLA may also be used, preferably L-PLA, either alone or in combination with PGA. Polycarbonates, polyfumarates and caprolactones may also be used to make the implants of this invention.

A plasticizer may be incorporated in the biodegradable film to make it softer and more pliable for applications where direct contact with a contoured surface is desired.

The polymeric films of this invention can be formed and used as flat sheets, or can be formed into three-dimensional conformations or “shells” molded to fit the contours of the tissue site into which the film is inserted.

To make the polymeric films of this invention, a suitable polymeric material is selected, depending on the degradation time desired for the film. Selection of such polymeric materials is known to the art. A lower molecular weight, e.g., around 20,000 daltons, 50:50 or 55:45 PLA:PGA copolymer is used when a shorter degradation time is desired. To ensure a selected degradation time, the molecular weights and compositions may be varied as known to the art.

Polymeric films of this invention may be made by dissolving the selected polymeric material in a solvent known to the art, e.g., acetone, chloroform or methylene chloride, using about 20 mL solvent per gram of polymer. The solution is then degassed, preferably under gentle vacuum to remove dissolved air and poured onto a surface, preferably a flat non-stick surface such as BYTAC (Trademark of Norton Performance Plastics, Akron, Ohio) non-stick coated adhesive-backed aluminum foil, glass or TEFLON™ non-stick polymer. The solution is then dried, preferably air-dried, until it is no longer tacky and the liquid appears to be gone. The known density of the polymer may be used to back-calculate the volume of solution needed to produce a film of the desired thickness.

Films may also be made by heat pressing and melt forming/drawing methods known to the art. For example, thicker films can be pressed to form thinner films, and can be drawn out after heating and pulled over forms of the desired shapes, or pulled against a mold by vacuum pressure.

The amount of the composition of the present invention to be incorporated into the polymeric films of this invention is an amount effective to show a measurable effect in treating hypercholesterolemia and hypoglycemia. The composition of the present invention can be incorporated into the film by various techniques such as by solution methods, suspension methods, or melt pressing.

Solid implants comprising the composition of the present invention can also be made into various shapes other than films by injection molding or extrusion techniques. For example, the implant can comprise a core material such as ethylene/vinyl acetate copolymer, and a vinyl acetate content of 20% by weight or more and which functions as a matrix for the composition of the present invention, in a quantity which is sufficient for a controlled release of the composition of the present invention, and a membrane which encases the core material and also consists of EVA material and an acetate content of less than 20% by weight. The implant can be obtained, for example, by means of a co-axial extrusion process, a method in which the two EVA polymers are extruded co-axially with the aid of a co-axial extrusion head. The co-axial extrusion process is art known per se so that it will not be gone into further within the scope of this description.

3. Transdermal Patch Device

Transdermal delivery, involves delivery of a therapeutic agent through the skin for distribution within the body by circulation of the blood. Transdermal delivery can be compared to continuous, controlled intravenous delivery of a drug using the skin as a port of entry instead of an intravenous needle. The therapeutic agent passes through the outer layers of the skin, diffuses into the capillaries or tiny blood vessels in the skin and then is transported into the main circulatory system.

Transdermal patch devices which provide a controlled, continuous administration of a therapeutic agent through the skin are well known in the art. Such devices, for example, are disclosed in U.S. Pat. Nos. 4,627,429; 4,784,857; 5,662,925; 5,788,983; and 6,113,940, which are all incorporated herein by reference. Characteristically, these devices contain a drug impermeable backing layer which defines the outer surface of the device and a permeable skin attaching membrane, such as an adhesive layer, sealed to the barrier layer in such a way as to create a reservoir between them in which the therapeutic agent is placed. In one embodiment of the present invention a formulation of the composition of the present invention is introduced into the reservoir of a transdermal patch.

4. Kits

The present invention also provides a kit comprising the therapeutic composition of the present invention and a suitable excipient as described herein and a set of instructions, generally written instructions, although electronic storage media (e.g., magnetic diskette or optical disk) containing instructions are also acceptable, relating to the use and dosage of the therapeutic composition of the present invention for the intended treatment. The instructions included with the kit generally include information as to dosage, dosing schedule, and route of administration for the intended treatment. The containers of the therapeutic composition of the present invention may be unit doses, bulk packages (e.g., multi-dose packages) or sub-unit doses.

The invention is further illustrated by the following non-limited examples. All scientific and technical terms have the meanings as understood by one with ordinary skill in the art. The specific examples which follow illustrate the methods in which the compositions of the present invention may be prepared and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### EXAMPLE 1

Tablets comprising a composition of the present invention are prepared as set out in Table III below:

TABLE III

| Ingredient | amt/cap | | Function |
|---|---|---|---|
| Chromax® (chromium picolinate) | 400 | mcg | Active |
| Policosanol | 20 | mg | Active |
| Calcium phosphate | 261.7 | mg | Base |
| Cellulose | 49.4 | mg | Tablet coating agent |
| Stearic acid | 23.8 | mg | Lubricant |
| Magnesium stearate | 6.8 | mg | Lubricant |
| Silicon dioxide | 9.4 | mg | Diluent |

It is therefore believed that the present invention provides an oral antihyperlipidemic and antihypoglycemic composition of policosanol and chromium and/or chromium salts, which is effective in increasing HDL cholesterol levels and lean body mass while reducing triglycerides, serum cholesterol levels, and blood glucose levels and a method of lowering cholesterol and glucose levels by employment of such an oral pharmaceutical or dietary supplement composition, or by the simultaneous oral administration of the ingredients thereof, all having the highly advantageous characteristics and effects as more fully set forth in the foregoing.

The foregoing description is considered as illustrative only of the principles of the invention. Further, since numerous modifications and changes will readily occur to those skilled in the art, it is not desired to limit the invention to the exact construction and process as described above. Accordingly, all suitable modifications and equivalents may be resorted to falling within the scope of the invention as defined by the claims that follow. The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

The embodiments of the invention in which an exclusive property or privilege is claimed are defined as follows:

1. A composition comprising policosanol and chromium and/or chromium salts.

2. The composition of claim 1, wherein said policosanol comprises a mixture of straight chain primary aliphatic alcohols from 20 to 36 carbons in length.

3. The composition of claim 2, wherein said mixture of straight chain primary aliphatic alcohols includes:

| | |
|---|---|
| 1-eicosanol (C-20) | 0-5% |
| 1-docosanol (C-22) | 0-5% |
| 1-tetracosanol (C-24) | 0-30% |
| 1-hexacosanol (C-26) | 5-30% |
| 1-heptacosanol (C-27) | 0-5% |
| 1-octacosanol (C-28) | 5-80% |
| 1-nonacosanol (C-29) | 0-5% |
| 1-triacontanol (C-30) | 5-40% |
| 1-dotriacontanol (C-32) | 1-25% |
| 1-tetratriacontanol (C-34) | 0-7% |
| 1-hexatriacontanol (C-36) | 0-5%. |

4. The composition of claim 2, wherein said mixture of straight chain primary aliphatic alcohols includes:

| | |
|---|---|
| 1-eicosanol (C-20) | 0-5% |
| 1-docosanol (C-22) | 0-5% |
| 1-tetracosanol (C-24) | 12-27% |
| 1-hexacosanol (C-26) | 13-28% |
| 1-heptacosanol (C-27) | 0-5% |
| 1-octacosanol (C-28) | 15-25% |
| 1-triacontanol (C-30) | 25-40% |
| 1-dotriacontanol (C-32) | 5-15% |
| 1-tetratriacontanol (C-34) | 0-5%. |

5. The composition of claim 1, wherein said chromium and/or chromium salt is selected from the group consisting of chromium, chromium picolinate (chromium, tris (2-pyridinecarboxylato-N(1), O(2))-(9CI)), chromium chloride, chromium nicotinate, chromium polynicotinate, chromium acetate, trivalent chromium, high-chromium yeast, chromium 2-pyridine-carboxylate, chromium tripicolinate, 2-pyridinecarboxylic acid-chromium salt, and tris(picolinato)chromium.

6. The composition of claim 1, further comprising a pharmaceutically acceptable carrier, excipient or dilutant.

7. The composition of claim 6, in the form of a capsule, tablet, liquid or powder.

8. A method for treating or preventing metabolic syndrome, hypercholesterolemia related diseases, and/or hypoglycemia related diseases, which comprises administering a pharmaceutically effective amount of a composition comprising policosanol and chromium and/or chromium salts to a human or mammal.

9. A method for reducing total cholesterol, LDL-cholesterol and blood glucose and increasing HDL-cholesterol levels and lean body mass, which comprises administering a pharmaceutically effective amount of a composition comprising policosanol and chromium and/or chromium salts to a human or mammal.

10. A method for lowering LDL-cholesterol, total cholesterol, blood glucose levels, increasing HDL-cholesterol, increasing lean body mass, and improving LDL-cholesterol/HDL-cholesterol ratio which comprises administering a composition comprising policosanol and chromium and/or chromium salts in a pharmaceutically acceptable amount to an individual in need thereof.

11. A method for lowering blood glucose levels which comprises administering a composition comprising policosanol and chromium and/or chromium salts in a pharmaceutically acceptable amount to an individual in need thereof.

12. The composition of claim 1 wherein said policosanol comprises at least one higher primary aliphatic alcohol selected from stright chain primary aliphatic alcohols having 20 to 36 carbon atoms, and said chromium and/or chromium salt is selected from the group consisting of chromium, chromium picolinate (chromium, tris (2-pyridinecarboxylato-N(1), O(2))-(9CI)), chromium chloride, chromium nicotinate, chromium polynicotinate, chromium acetate, trivalent chromium, high-chromium yeast, chromium 2-pyridine-carboxylate, chromium tripicolinate, 2-pyridinecarboxylic acid-chromium salt, and tris(picolinato)chromium wherein said composition is further characterized by a combination of policosanol and chromium and/or chromium salts in a quantitative ratio from 100:1 to 0.01:1 by weight.

13. The composition of claim 12 wherein said policosanol comprises 1-tetracosanol, 1-hexacosanol, 1-octacosanol, 1-triacontanol, 1-dotriacontanol and 1-tetratriacontanol, said composition is further characterized by a combination of policosanol and chromium and/or chromium salts in a quantitative ratio from 10:1 to 0.10:1 by weight.

14. The composition of claim 13, wherein said policosanol has the following quantitative composition:

| | |
|---|---|
| 1-docosanol (C-22) | 0-5 wt % |
| 1-tetracosanol (C-24) | 0-30 wt % |
| 1-hexacosanol (C-26) | 5-30 wt % |
| 1-heptacosanol (C-27) | 5-10 wt % |
| 1-octacosanol (C-28) | 10-20 wt % |
| 1-nonacosanol (C-29) | 0-5 wt % |
| 1-triacontanol (C-30) | 5-40 wt % |
| 1-dotriacontanol (C-32) | 1-25 wt % |
| 1-tetratriacontanol (C-34) | 0 7 wt %; |

and said composition is further characterized by a combination of policosanol and chromium and/or chromium salts in a quantitative ratio from 3:1 to 0.33:1 by weight.

15. A method of treating metabolic syndrome, hypercholesterolemia and hypoglycemia related diseases, total cholesterol, LDL-cholesterol, LDL/HDL ratio, triglycerides, coronary heart disease (heart attacks and strokes), inflammation, deep-vein thrombosis, immunoregulatory diseases, cardiovascular diseases, obesity, insulin resistance, dyslipidemia, raised blood pressure, fatigue, premenstrual syndrome, anxiety, depression and/or neurodegenerative disorders, and/or raise HDL cholesterol and/or lean body mass in a patient, comprising delivering to said patient a composition comprising policosanol and chromium and/or chromium salts in an amount effective to reduce and/or prevent metabolic syndrome, hypercholesterolemia and hypoglycemia related diseases, total cholesterol, LDL-cholesterol, LDL/HDL ratio, triglycerides, coronary heart disease (heart attacks and strokes), inflammation, deep-vein thrombosis, immunoregulatory diseases, cardiovascular diseases, obesity, insulin resistance, dyslipidemia, raised blood pressure, fatigue, premenstrual syndrome, anxiety, depression and/or neurodegenerative disorders, and/or raise HDL cholesterol and/or lean body mass in an individual in need thereof, wherein said composition is delivered to said patient as a controlled/sustained/extended/prolonged release composition.

16. The method of claim 15, wherein said controlled/sustained/extended/prolonged release composition comprises a flowable thermoplastic polymer composition comprising a biocompatible polymer, a biocompatible solvent, policosanol and chromium and/or chromium salts and said controlled/sustained/extended/prolonged release composition is delivered to a bodily tissue or fluid in said patient, wherein the amounts of the polymer and the solvent are effective to form a biodegradable polymer matrix containing policosanol and chromium and/or chromium salts in situ when said composition contacts said bodily fluid tissue or fluid.

17. The method of claim 16, wherein said polymer is a poly(alkylene glycol) or a polysaccharide.

18. The method of claim 15, wherein the composition further comprises a controlled/sustained/extended/prolonged release additive.

19. The method of claim 16, wherein said biocompatible polymer is selected from the group consisting of polylactides, polyglycolides, polyanhydrides, polyorthoesters, polycaprolactones, polyamides, polyurethanes, polyesteramides, polydioxanones, polyacetals, polyketals, polycarbonates, polyorthocarbonates, polyphosphazenes, polyhydroxybutyrates, polyhydroxyvalerates, polyalkylene oxalates, polyacrylates, polyalkylene succinates, poly(malic acid), poly(amino acids) and copolymers, terpolymers, cellulose diacetate, ethylene vinyl alcohol, and copolymers and combinations thereof.

20. The method of claim 16, wherein said biodegradable polymer matrix releases policosanol and chromium and/or chromium salts by diffusion, erosion, or a combination of diffusion or erosion as the polymer matrix biodegrades in said patient.

21. The method of claim 16, wherein said policosanol and chromium and/or chromium salts are added to said polymer composition prior to administration such that said solid polymer matrix further contains said policosanol and chromium and/or chromium salts.

22. The method of claim 15, wherein said controlled/sustained/extended/prolonged release composition is in film form.

23. The method of claim 22, wherein said film comprises polylactic acid, polyglycolic acid and mixtures and copolymers thereof.

24. The method of claim 15, wherein said controlled/sustained/extended/prolonged release is in tablet form.

25. A kit comprising: a first container comprising a controlled/sustained/extended/prolonged release formulation of policosanol and chromium and/or chromium salts, said formulation comprising an amount of policosanol and chromium and/or chromium salts effective to treat or reduce and/or prevent metabolic syndrome, hypercholesterolemia and hypoglycemia related diseases, total cholesterol, LDL-cholesterol, LDL/HDL ratio, triglycerides, coronary heart disease (heart attacks and strokes), inflammation, deep-vein thrombosis, immunoregulatory diseases, cardiovascular diseases, obesity, insulin resistance, dyslipidemia, raised blood pressure, fatigue, premenstrual syndrome, anxiety, depression and/or neurodegenerative disorders, and/or raise HDL cholesterol and/or lean body mass.

26. The kit of claim 25, further comprising a puncture needle or catheter.

27. An article of manufacture comprising:
(a) a stent body comprising a surface; and
(b) a coating comprising at least one layer disposed over at least a portion of the stent body, wherein the said layer comprises a polymer film having at least one biologically active agent dispersed therein.

28. The article of manufacture of claim 27, wherein said biologically active agent is policosanol and chromium and/or chromium salts.

29. A method of reducing LDL-cholesterol, triglyceride and/or blood glucose levels comprising:
administering to a mammal a pharmaceutical composition in an amount that inhibits VLDL triglyceride output and inhibits the conversion of acetate to acetyl CoA while not raising uric acid levels, glucose levels and/or homocysteine levels.

30. The method of claim 29, wherein the administration of said composition further comprises raising HDL-cholesterol levels and/or lean body mass.

31. The method of claim 29, wherein the triglyceride output is inhibited by chromium and/or chromium salts.

32. The method of claim 29, wherein the blood glucose levels are reduced by chromium and/or chromium salts.

33. The method of claim 29, wherein the conversion of acetate to acetyl CoA is inhibited by policosanol.

34. The method of claim 29, wherein the lean body mass is raised by chromium and/or chromium salts.

35. The composition of claim 4, wherein said chromium and/or chromium salt is selected from the group of chromium, chromium picolinate (chromium, tris (2-pyridinecarboxylato-N(1), O(2))-(9CI)), chromium chloride, chromium nicotinate, chromium polynicotinate, chromium acetate, trivalent chromium, high-chromium yeast, chromium 2-pyridine-carboxylate, chromium tripicolinate, 2-pyridinecarboxylic acid-chromium salt, and tris(picolinato)chromium.

36. The composition of claim 1, wherein said chromium and/or chromium salts are administered in a daily dose within a range from 0.05 mcg/day to 2,000 mcg/day.

37. The composition of claim 1, wherein said chromium and/or chromium salts are selected from the group consisting of chromium, chromium picolinate (chromium, tris (2-pyridinecarboxylato-N(1), O(2))-(9CI)), chromium chloride, chromium nicotinate, chromium polynicotinate, chromium acetate, trivalent chromium, high-chromium yeast, chromium 2-pyridine-carboxylate, chromium tripicolinate, 2-pyridinecarboxylic acid-chromium salt, and tris(picolinato)chromium administered in a daily dose in the range of 0.1-1,000 mcg/day.

38. The composition of claim 1, wherein said chromium is administered in a daily dose in the range of 0.2-70 mcg/day.

39. The composition of claim 1, wherein said chromium salts are selected from the group consisting of chromium picolinate (chromium, tris (2-pyridinecarboxylato-N(1), O(2))-(9CI)), chromium chloride, chromium nicotinate, chromium polynicotinate, chromium acetate, trivalent chromium, high-chromium yeast, chromium 2-pyridine-carboxylate, chromium tripicolinate, 2-pyridinecarboxylic acid-chromium salt, and tris(picolinato)chromium administered in a daily dose in the range of 100-1,000 mcg/day.

40. A controlled/sustained/extended/prolonged release preparation comprising a pharmaceutically active mixture of policosanol and chromium and/or chromium salts.

41. A transdermal preparation designed to administer a pharmaceutically effective amount of policosanol and chromium and/or chromium salts into the blood stream.

42. The transdermal preparation of claim 41, wherein the policosanol and chromium and/or chromium salts are present in a concentration sufficient that when applied to the skin a pharmaceutically effective steady state plasma concentration in the patient of said chromium and/or chromium salts is produced.

43. A transdermal delivery system for application to the skin of a patient, comprising:
(a) a drug impermeable backing layer;
(b) an adhesive layer;
(c) a drug permeable membrane, wherein the membrane is positioned relative to the backing layer so as to form at least one drug reservoir compartment between the membrane and the backing layer; and
(d) a composition comprising policosanol and chromium and/or chromium salts contained within the drug reservoir compartment in a concentration sufficient such that the transdermal delivery system has an input rate when applied to the skin sufficient to produce a pharmaceutically effective steady state plasma concentration in the patient.

44. The method of claim 15, wherein said controlled/sustained/extended/prolonged release composition comprises applying a transdermal delivery system containing a mixture of policosanol and chromium and/or chromium salts to the skin of a patient and maintaining the transdermal delivery system in contact with the skin for a time sufficient to provide a pharmaceutically effective steady state plasma concentration in the patient.

45. A subcutaneous implant comprising policosanol and chromium and/or chromium salts.

46. The subcutaneous implant of claim 44 wherein said implant is effective to release levels of policosanol and chromium and/or chromium salts over an extended period of time when subcutaneously implant in a human or animal in need thereof.

47. A method for administering policosanol and chromium and/or chromium salts to a human or animal which comprises subcutaneously implanting either a biodegradeable or nonbiodegradable polymer comprising a mixture of policosanol and chromium and/or chromium salts.

48. The method of claim 15, wherein said controlled/sustained/extended/prolonged release composition comprises administering subcutaneously to the patient a mixture of policosanol and chromium and/or chromium salts.

49. The methods of claims 8, 9, 10, and 11, further comprising administering asprin.

50. The method of claim 49, wherein said aspirin is administered in a dose in the range of 162-325 mg.

51. The composition of claim 1, further comprising aspirin.

52. The composition of claim 1, further comprising aspirin administered in a dose in the range of 162-325 mg.
